(19) United States
(12) Patent Application Publication
(10) Pub. No.: US 2022/0370665 A1
(43) Pub. Date: Nov. 24, 2022

(54) DECONTAMINATION SYSTEMS

(71) Applicant: Battelle Memorial Institute, Columbus, OH (US)

(72) Inventors: Lindy Espina DEJARME, Columbus, OH (US); Matthew S. YOUNG, Upper Arlington, OH (US); David CHASE, Gahanna, OH (US); Cynthia DODARO, Grove City, OH (US); Derek M. MICHELIN, Plymouth, MA (US); William RICHTER, Hilliard, OH (US); Gary A. STICKEL, London, OH (US); Andrew V. SWEENEY, Columbus, OH (US); Krenar SHQAU, Westerville, OH (US)

(21) Appl. No.: 17/751,003

(22) Filed: May 23, 2022

Related U.S. Application Data
(60) Provisional application No. 63/192,053, filed on May 23, 2021.

(51) Int. Cl. A61L 2/22; A61L 2/06; A61L 2/24

(52) U.S. Cl. CPC A61L 2/22; A61L 2/06; A61L 2/24; A61L 2202/122; A61L 2202/14; A61L 2202/15; A61L 2101/34

(57) ABSTRACT

The present disclosure provides decontamination system that includes a decontamination chamber for sealably holding one or more items for decontamination. The system also includes a decontamination unit comprising: a controllable air heater and humidity control unit to controllably heat an ambient air source and to control a temperature and humidity of air within a decontamination chamber; a controllable vaporizer unit to controllably vaporize one or more decontamination agents by combining the controllably heated ambient air with the one or more decontamination agents; and controller circuitry to control a controllable air heater and humidity control unit to heat the ambient air to a selected level, and to maintain a selected heat and humidity within the decontamination chamber, and to control the controllable vaporizer unit to provide a selected flow rate of the one or more decontamination agents into the decontamination chamber.

This application claims the benefit of U.S. Provisional Application Ser. No. 63/192,053, filed May 23, 2021, which is hereby incorporated by reference in its entirety.

### TECHNICAL FIELD

The present disclosure relates to decontamination systems.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 illustrates a decontamination system in accordance with several embodiments of the present disclosure;

FIG. 2 illustrates a decontamination system in accordance with one embodiment of the present disclosure;

FIG. 3 illustrates a stationary decontamination system according to one embodiment of the present disclosure; and

FIG. 4 illustrates a portable decontamination system according to one embodiment of the present disclosure.

Although the following Detailed Description will proceed with reference being made to illustrative embodiments, many alternatives, modifications and variations thereof will be apparent to those skilled in the art.

### DETAILED DESCRIPTION

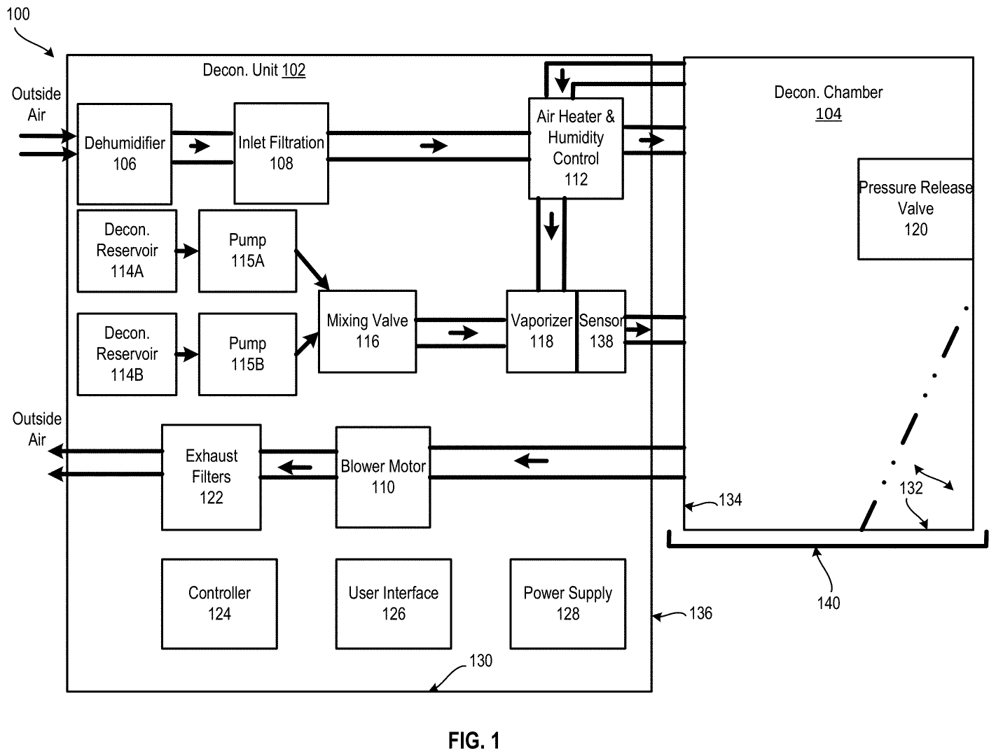

FIG. 1 illustrates a decontamination system 100 in accordance with several embodiments of the present disclosure. As a general overview, the decontamination system 100 may be used in a warfare environment to enable field decontamination of battle weapons, gear, vehicles, electronic equipment (e.g., cell phones, radios, computers, laptops, etc.), etc. In addition, the decontamination system may be used in a civilian environment such as offices, hospitals, industrial warehouses, etc. The decontamination system 100 of the present disclosure is configured to decontaminate a wide variety of contaminants such chemical warfare agents (CWA) such as vesicants as HD and nerve agents such as VX, biological warfare agents (BWA) such as Yersinia pestis, SARS-CoV-2, and Bacillus anthracis, toxic industrial chemicals (TIC) such as carbon disulfide and parathion, etc.

The decontamination system 100 includes a self-contained decontamination unit 102 and a decontamination chamber 104. The decontamination unit 102 includes a dehumidifier unit 106 generally configured to remove moisture content, at least partially, from outside air being drawn into the decontamination unit 102. The dehumidifier unit 106 may be controllable to achieve a desired humidity content of outside air. For example, if the outside air has a humidity content of greater than 50%, the dehumidifier unit 106 may be controlled to achieve a humidity level of less than 50%. The decontamination unit 102 also includes an inlet filtration unit 108 generally configured to filter particulates and/or contaminants from outside air being drawn in the unit 102. The inlet filtration unit 108 may include one or more of, for example, carbon filtration, High Efficiency Particulate Air (HEPA) filtration, chemical reagent filtration, etc. The decontamination unit 102 also includes a controllable blower motor 110 generally configured to draw outside air into the dehumidifier 106/filtration 108. The speed of the motor 110 may be controlled to control an air flow rate.

The decontamination unit 102 also includes a controllable air heater and humidity control unit 112 generally configured to heat air being drawn into the unit 102, heat air contained within the decontamination chamber 104, and control a humidity level within the decontamination chamber 104. In some embodiments, the controllable air heater and humidity control unit 112 is configured to maintain an air temperature within the chamber 104 at around 50 degrees Celsius, and maintain a humidity of the air within the chamber 104 at about 50% humidity. Of course, these are only examples of the temperature and humidity maintained in the chamber 104, and in other embodiments, the temperature and/or humidity may be controlled based on, for example, the duration of decontamination time, the nature of the contaminant(s), the optimal operating environments for decontamination agents, prevention of condensation of decontamination agents, etc. The controllable air heater and humidity control unit 112 may also be configured to receive air from within the decontamination chamber 104 to provide feedback control over air temperature and humidity within the chamber 104. The controllable air heater and humidity control unit 112 is also configured to delivery temperature and humidity controlled air to a vaporizer unit 118, described below.

The decontamination unit 102 also includes a first decontamination agent reservoir 114A to hold a first decontamination agent to decontaminate items contained in the chamber 104. The first decontamination agents may be selected for a given contaminant and may include known aqueous decontamination agents such as hydrogen peroxide, ammonia, etc. The decontamination unit 102 may also include a second decontamination agent reservoir 114B to hold a second decontamination agent to decontaminate items contained in the chamber 104. The second decontamination agent may be selected for a given contaminant and may include known aqueous decontamination agents such as hydrogen peroxide, ammonia, etc., and may also include a different concentration of the first decontamination agent. Controllable pumps 115A and 115B are coupled to the decontamination reservoirs 114A and 114B, respectively, to control the flow of decontamination agents into the decontamination chamber 104. In some embodiments, the pumps 115A and 115B may be controlled to reverse the flow of agents back into the reservoirs 114A and/or 114B. For certain contaminants, two or more decontamination agents may be mixed to provide greater decontamination ability. For example, hydrogen peroxide and ammonia may be mixed for some decontamination tasks. Accordingly, the decontamination unit 102 also includes a controllable mixing valve 116 generally configured to control a mixture of two or more decontamination agents and/or control a flow volume of one or more decontamination agents. To enable more complete decontamination of items in the chamber 104, the decontamination unit 102 also includes a controllable vaporizer to vaporize one or more decontamination agents by mixing heated air from air heater unit 112 with the one or more decontamination agents to form suspended droplets of decontamination agent(s). Senser 138 is generally configured to detect a flow rate of the vaporized decontamination agent(s) and/or to detect the presence and/or concentration of the decontamination agent(s).

In some embodiments, the decontamination agent(s) may be selected from a wide range of known and/or after developed chemicals. For example, the decontamination agent may include high concentration peroxide (e.g., 35% aqueous H2O2) and/or low concentration peroxide (e.g., 3-7% aqueous H2O2). The decontamination agent may also include 4% aqueous ammonium hydroxide (NH3) and/or anhydrous ammonia in a pressurized cylinder, etc. Advantageously, the use of low concentration decontamination agents may enable unrestricted national and/or international transport of the decontamination unit 102, increased global availability of decontamination agents and/or safer operation and disposal of decontamination agents by reducing chemical contact and down-the-drain hazards. In some embodiments, one or more decontamination agents may be formulated in the field, for example, using low concentration agents. For example, low concentration H₂O₂ (e.g., 3% or lower) may be used to form a higher concentration H₂O₂ (e.g., 35%, etc.) using fractional distillation techniques. In such an embodiment, a freezer unit (e.g., a U85-13 freezer unit manufactured by So-Low Environment Equipment, or similar, not shown) may be included to freeze low concentration H2O2 in successive cycles to remove water. In other embodiments, NH3 may be prepared in the field using NH4Cl, a solid salt, and Ca(OH)2 (or NaOH), and/or other solid-form reactions that enable field preparation of aqueous ammonia.

To maintain a relatively constant pressure within the chamber 104, the chamber 104 may also include controllable pressure release valve 120 to “bleed” off pressure within the chamber 104 when the pressure exceeds a selected threshold. In one embodiment, the pressure relief valve 120 may be controlled so that the decontamination chamber 104 has a slight negative pressure (e.g., −0.5 to −2″ water column) within the chamber 104, compared to ambient pressure outside of the chamber 104. The decontamination unit 102 may also include a controllable blower motor 110 in fluid communication with the chamber 104 to cause airflow to the outside of the chamber 104. If the pressure within the chamber 104 drops below a selected negative pressure, the pressure relieve valve 120 may automatically and/or controllable open to draw outside air into the chamber 104. To prevent contaminants and/or decontamination agent(s) from exiting the unit 102, the unit 102 may also include one or more exhaust filters 122 generally configured to reduce or eliminate contaminants and/or decontamination agent(s) from an exhaust stream from the chamber 104. In some embodiments, the exhaust filters 122 may include a two-stage filtration unit having a charcoal activated first stage and a HEPA filter second stage. In some embodiments, a carbon (charcoal) filter may be used to remove NH3 from the exhaust and a catalytic converter (e.g., Iron-based catalysts) may be used to remove peroxide from the exhaust. Of course, these are only examples of the types of filters that may form the exhaust filters 122, and in other embodiments, the exhaust filters 122 may be selected based on, for example, required exit air quality tolerance, the contaminants within the chamber 104, the selected decontamination agents, etc.

It should be noted that surfaces within the decontamination unit 102 that may come in contact with contaminants and/or decontamination agent(s) may be formed of inert and/or corrosion resistant materials such as stainless steel, Teflon, etc. Such surfaces may include, for example, tubing/ducts that connect the various units described above, contact surfaces of the air heater 112, mixing valve, pressure release valve 120, etc. The decontamination unit 102 may be packaged as a self-contained unit within a closed structure 130. The closed structure may be formed of chemical, temperature and/or heat resistant materials such as stainless steel, etc. The decontamination unit 102 may scalable, depending on a size of the chamber 104, desired flow rate, desired volume of decontamination agent(s), etc.

The decontamination chamber 104 is generally a sealable enclosed chamber to hold one or more items for decontamination. The decontamination chamber 104 may be sized for a given decontamination task. For example, the chamber 104 may conform to industry standard container sizes (e.g., ISO standards, etc.), for example, having a length of 40 ft., 20 ft., 10 ft. 8 ft., etc. The decontamination chamber 104 may include a door or sealable opening 132 to enable items to be placed in, and removed from the chamber 104. The door or sealable opening 132 may include latching/locking mechanisms (not shown) and one or more seals around the periphery thereof to provide an airtight seal of the chamber 104. Such seals may be selected to be inert to decontamination agent(s) within the chamber 104. The chamber 104 may include one or more air vents to receive the heated air from the air heater 112, one or more registers (not shown) to provide feedback air flow to the air heater unit 112, and one or more nozzles and/or misters (not shown) to introduce the vaporized decontamination agent(s) from the vaporizer 118 In addition, the chamber 104 may include one or more controllable fans (not shown) to provide air/decontamination agent movement within the chamber 104. The chamber 104 may also include an exhaust port to communicate with the pressure relief valve 120 to maintain a selected pressure in the chamber. The interior surfaces of the chamber 104 may be formed of inert and/or corrosion resistant materials such as stainless steel, Teflon, etc. The chamber 104 may also include reinforcement structures, depending on the size and/or weight of items to be placed therein. The chamber 104 may also include one or more removable walls/barriers to reduce the volume of the chamber, e.g., for smaller items to be decontaminated.

In some embodiments, the chamber 104 may be partially or fully enclosed by a controllable heating wrap (e.g., insulating blanket) to enable the interior of the chamber to be heated to a desired temperature, and also to prevent and/or control condensation of decontamination agents. The chamber 104 may also include insulated walls, roof and floor to enable the chamber to hold temperature for a longer period of time.

In some embodiments, the decontamination unit 102 and the chamber 104 may be formed as a unitary structure. In other embodiments, the decontamination unit 102 and the chamber 104 may be separate, modular units. In such embodiments, the decontamination unit 102 and the chamber 104 may each include interface structures 134/136 to enable a tight seal between the decontamination unit 102 and the chamber 104. The interface structures 134/136 may include, for example, locking mechanisms, seals, latching mechanisms, friction fit couplings, etc. Such embodiments may enable the decontamination unit 102 and/or the chamber 104 to be portable. The decontamination unit 102 may also include controller circuitry 124 generally configured to exchange commands and data with one or more of the controllable units described above, based on feedback information received from, for example, sensor 138, dehumidifier 103, air heater and humidity control 112, mixing valve 116, pressure release valve 120, etc. The decontamination unit 102 may also include a user interface 126 to enable a user to control a decontamination process by controlling air temperature, humidity, vaporization levels, flow control, mixing levels, etc. The user interface 126 may also provide emergency power off control over the decontamination unit 102, etc. The decontamination unit 102 may also include one or more power supplies 128 (e.g., battery/solar powered power supply, gas powered power supply, diesel powered power supply, etc.) to provide power to one or more units described above. In some embodiments, the decontamination unit 102 may also include communications circuitry (e.g., cellular communications circuitry, WiFi/Ethernet communications circuitry, etc., not shown) to exchange commands and data with a remote link partner to enable, for example, communication of operational status of the unit 102, remote control of the unit 102, etc.

By way of example, the controller circuitry 124 may be configured to execute various operational modes to control decontamination, where one or more modes may be selected depending on the contaminant and/or the size/shape of the item to be decontaminated. A first mode may include a heat only mode. In this mode, the controller circuitry 124 may control the air heater and humidity control 112 to heat the decontamination chamber to a selected temperature (e.g., 50-60 degrees Celsius). This first mode may be effective to decontaminate biological agents (e.g. Yersinia pestis or SARS-CoV-2) and selected chemical agents (e.g., sarin, isopropyl methylphosphonofluoridate, etc.). In this heat only mode, a decontamination time may be selected to ensure proper decontamination (and such a time may be longer than when using decontamination agents) (e.g. heat-only cycle 120 to 240 minutes vs. heat+H2O2 cycle 10-60 minutes). A second mode may include a peroxide and heat mode. In this second mode, the controller circuitry 124 mode may control the air heater and humidity control 112 to heat the decontamination chamber to a selected temperature (e.g., 50-60 degrees Celsius), and also control the mixing valve 116 and/or vaporizer 118 for a selected flow control of H2O2 into the chamber 104. This mode is effective against multiple biological agents (e.g. Yersinia pestis, SARS-CoV-2, and Bacillus anthracis) including biological spores, and this second mode may comply or be compatible with industry standard methodology for decontamination of sensitive equipment (such as cellular phones, radios and computers) to medical device sterilization standards greater than 6 log or 99.9999% spore reduction achieve in less than an hour of decontamination (and faster than heat only).

A third mode includes a condensing peroxide mode. In this mode, heating the chamber 104 may be omitted to allow condensation of H2O2. In this mode, H2O2 is injected until the chamber and contents reach saturation point at which time the vaporized H2O2 condenses onto the surfaces of all materials, including the chamber skin, inside the chamber. This mode is applicable to multiple biological agents (e.g. Yersinia pestis, SARS-CoV-2, and Bacillus anthracis) including biological spores with decontamination achieve at greater than 6 log or 99.9999% reduction in less than an hour of decontamination and faster than heat only.

A fourth mode includes using H2O2, NH3 and heat in a non-condensing cycle. In this mode, the chamber 104 may be heated to approximately 50 degrees Celsius, H2O2 concentrations up to 500 ppm, >30 ppm NH3, <60% humidity (which may be measured with a Vaisala HPP 270 or similar equipment). This mode is effective against chemical warfare (CW) agents (e.g., distilled sulfur mustard, mustard gas, pinacolyl methylphosphonofluoridate, soman, sarin, isopropyl methylphosphonofluoridate, etc.) and VX (e.g., venomous agent X, Ethyl N-2-diisopropylaminoethyl methylphosphonothiolate, etc.) without forming secondary hazards such as vesicant sulfone or EA-2192.

A fifth mode of decontamination operations includes using NH3 and heat, for example, 50 degrees Celsius and >30 PPM NH3. In this mode, ammonia is not considered a disinfectant for biological organisms but a good cleansing agent (hence a decontaminant) that moves materials from inaccessible areas to where decontamination/destruction can be implemented.

In some embodiments, the chamber 104 may be aerated after decontamination. Aeration may include venting the chamber with clean air until detection of decontamination agents falls below a selected concentration (e.g., below 1 PPM, etc.).

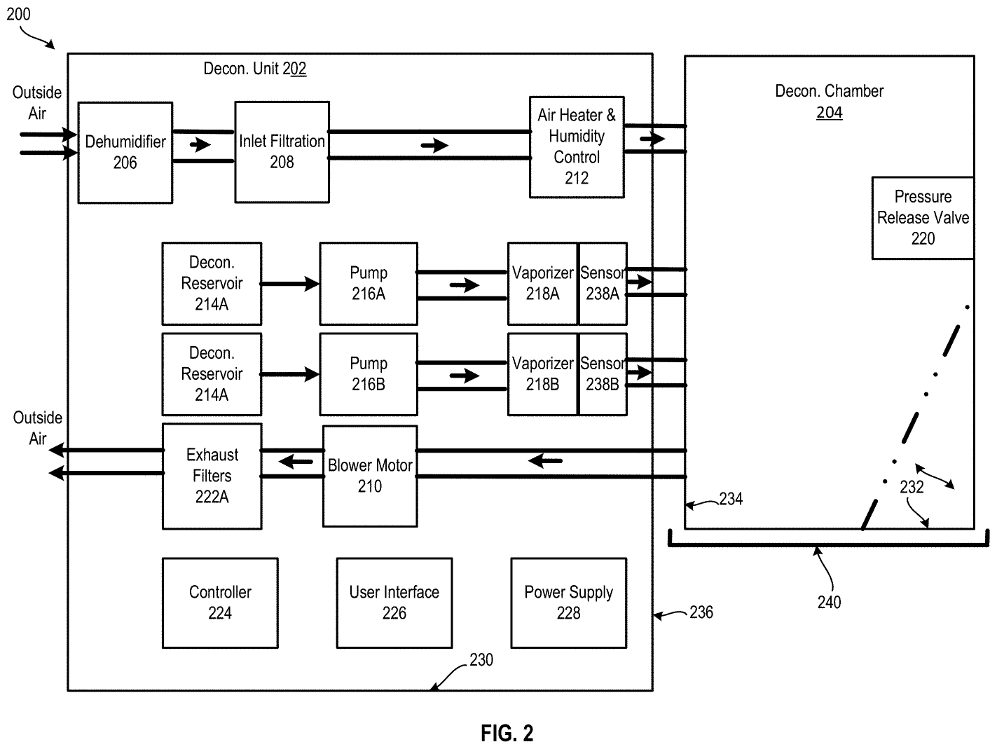

FIG. 2 illustrates a decontamination system 200 in accordance with several embodiments of the present disclosure. The decontamination system 200 of this embodiment is similar to the system 100 of FIG. 1, except that decontamination agents are provided with independent input paths. Using independent decontamination agent paths may enable, for example, more accurate sensing and flow control of decontamination agents (e.g., a single electrochemical sensor may be incapable of accurately measuring NH3 and H2O2 simultaneously). For example, the system 200 of FIG. 2 includes a first decontamination path that includes a first decontamination reservoir 214A, a first controllable flow control valve 216A, a first vaporizer unit 218A and a first sensor 238A. The first decontamination reservoir 214A may hold NH3 at a selected concentration, and the first sensor 238A may be an electrochemical sensor configured to sense the presence and/or concentration of NH3 (using, for example, a Draeger Polytron 7000 electrochemical sensor, or similar, etc.). The system 200 of FIG. 2 also includes a second decontamination path that includes a second decontamination reservoir 214B, a second controllable flow control valve 216B, a second vaporizer unit 218B and a second sensor 238B. The second decontamination reservoir 214B may hold H2O2 at a selected concentration, and the second sensor 238B may be an electrochemical sensor configured to sense the presence and/or concentration of H2O2 (using, for example, a Draeger Polytron 7000 electrochemical sensor, or similar, etc.). In another embodiment, sensors 238A and/or 238B may be replaced for direct measurement of NH3 and/or H2O2 inside the chamber 204, using for example mass spectrometry (e.g., a Bruker Spectrometer Omega5 or similar). Of course, the system 200 of FIG. 2 may be extended to include more than two independent decontamination agent paths. Other components illustrated in FIG. 2 (and operational modes thereof) are described above with reference FIG. 1, where the description of FIG. 1 applies to similarly-named parts of FIG. 2.

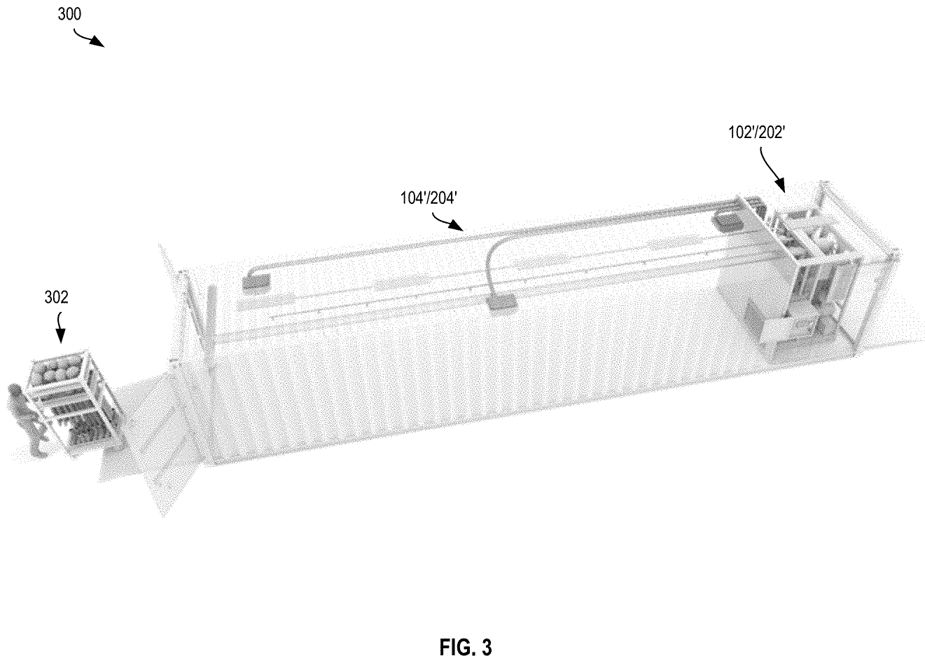

FIG. 3 illustrates a unitary decontamination system 300 according to one embodiment of the present disclosure. In this embodiment, the decontamination unit 102′/202′ and decontamination chamber 104′/204′ are formed as a single unit. As illustrated, the chamber 104′/204′ includes a sealable door to permit placement of one or more items 302 into the chamber for decontamination.

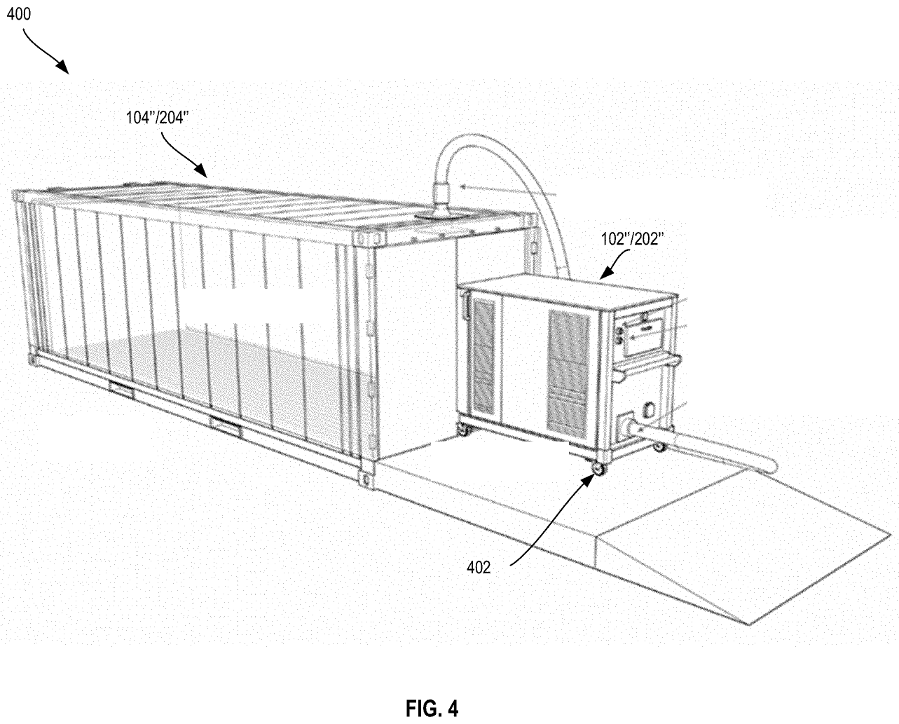

FIG. 4 illustrates a modular decontamination system 400 according to one embodiment of the present disclosure. In this embodiment, the decontamination unit 102″/202″ and decontamination chamber 104″/204″ are formed as separate units. In this embodiment, the decontamination unit 102″/202″ includes wheels 402 for portability.

Accordingly, there has been provided herein decontamination systems that offer significant advantages over conventional decontamination systems. For example, the decontamination systems described herein may include form factors to interface with common commercial shipping container equipment, transportation infrastructure, logistical centers, distribution channels, and/or shipping networks. For example, form factors include the ability to interface with tilt-bed trucks, flatbed trails, air freight equipment, maritime shipping equipment, etc.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuitry”, as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DST), field programmable gate array (FPGA), logic gates, registers, semiconductor device, chips, microchips, chip sets, etc.

Any of the operations described herein may be implemented in a system that includes one or more non-transitory storage devices having stored thereon, individually or in combination, instructions that when executed by circuitry perform one or more operations. Here, the circuitry may include any of the aforementioned circuitry including, for examples, one or more processors, ASICs, ICs, etc., and/or other programmable circuitry. Also, it is intended that operations described herein may be distributed across a plurality of physical devices, such as processing structures at more than one different physical location. The storage device includes any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software executed by a programmable control device.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification., as will be understood by those having skill in the art. The present disclosure should, therefore, be considered to encompass such combinations, variations, and modifications.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed:

1. A decontamination system, comprising:
a controllable air heater and humidity control unit to controllably heat an ambient air source and to control a temperature and humidity of air within a decontamination chamber;
a controllable vaporizer unit to controllably vaporize one or more decontamination agents by combining the controllably heated ambient air with the one or more decontamination agents; and
controller circuitry to control a controllable air heater and humidity control unit to heat the ambient air to a selected level, and to maintain a selected heat and humidity within the decontamination chamber, and to control the controllable vaporizer unit to provide a selected flow rate of the one or more decontamination agents into the decontamination chamber.

2. The decontamination system of claim 1, further comprising:
a controllable dehumidifier unit in fluid communication with the controllable air heater and humidity control unit to dehumidify the ambient air source to a selected humidity level;
an inlet filtration unit in fluid communication with the controllable dehumidifier unit to filter the ambient air; and
a controllable blower motor in fluid communication with the controllable dehumidifier unit to controllably draw the ambient air into the controllable dehumidifier unit and force the ambient air into the controllable air heater and humidity control unit.

3. The decontamination system of claim 1, further comprising:
a decontamination agent reservoir to hold the one or more decontamination agents; and
a controllable mixing valve for controlling a mixture of two or more decontamination agents.

4. The decontamination system of claim 1, further comprising:
user interface circuitry to enable a user to operate the system; and
power supply circuitry to supply power to at least the controllable air heater and humidity control unit, controllable vaporizer unit, and the controller circuitry.

5. The decontamination system of claim 1, further comprising:
a controllable pressure relief valve to maintain a selected pressure within the decontamination chamber; and
an exhaust port filtration unit to filer air exiting the decontamination chamber through the controllable pressure relief valve.

6. The decontamination system of claim 1, wherein the temperature and humidity of air within the decontamination chamber are controlled to be approximately 50 degrees Celcius and 50% humidity.

7. The decontamination system of claim 1, wherein the controllable air heater and humidity control unit, controllable vaporizer unit, and the controller circuitry are packaged into a closed structure, and wherein the closed structure is portable.

8. The decontamination system of claim 1, wherein the one or more decontamination agents are selected from an aqueous hydrogen peroxide solution and/or an aqueous ammonia solution.

9. The decontamination system of claim 1, wherein the controller circuitry to control the controllable air heater and humidity control unit to operate in a heat only mode wherein the flow rate of the one or more decontamination agents is controlled to be zero, and the decontamination chamber is heated to a selected temperature.

10. The decontamination system of claim 1, wherein the decontamination agent is aqueous H2O2, and wherein the controller circuitry to control the controllable air heater and humidity control unit and the controllable vaporizer to operate in a heat and H2O2 mode, wherein the flow rate of the H2O2 decontamination agent is controlled remove contaminants within the decontamination chamber, and the decontamination chamber is heated to approximately 50-60 degrees Celsius.

11. The decontamination system of claim 1, wherein the decontamination agent is aqueous NH3, and wherein the controller circuitry to control the controllable air heater and humidity control unit and the controllable vaporizer to operate in a condensing NH3 mode, wherein the flow rate of the NH3 decontamination agent is controlled remove contaminants within the decontamination chamber, and a temperature of the decontamination chamber is controlled to enable the NH3 to condense.

12. The decontamination system of claim 1, wherein the decontamination agents include aqueous H2O2 and aqueous NH3, and wherein the controller circuitry to control the controllable air heater and humidity control unit and the controllable vaporizer to operate in a heat, NH3 and H2O2 mode, wherein the flow rate of the H2O2 and NH3 decontamination agents are controlled remove contaminants within the decontamination chamber, and the decontamination chamber is heated to approximately 50 degrees Celsius.

13. The decontamination system of claim 1, wherein the decontamination agent is aqueous NH3, and wherein the controller circuitry to control the controllable air heater and humidity control unit and the controllable vaporizer to operate in a heat and NH3 mode, wherein the flow rate of the NH3 decontamination agent is controlled remove contaminants within the decontamination chamber, and a temperature of the decontamination chamber is heated to approximately 50 degrees Celsius.

14. A decontamination system, comprising:
a decontamination chamber for sealably holding one or more items for decontamination; and
a decontamination unit comprising:
a controllable air heater and humidity control unit to controllably heat an ambient air source and to control a temperature and humidity of air within a decontamination chamber;
a controllable vaporizer unit to controllably vaporize one or more decontamination agents by combining the controllably heated ambient air with the one or more decontamination agents; and
controller circuitry to control a controllable air heater and humidity control unit to heat the ambient air to a selected level, and to maintain a selected heat and humidity within the decontamination chamber, and to control the controllable vaporizer unit to provide a selected flow rate of the one or more decontamination agents into the decontamination chamber.

15. The decontamination system of claim 14, wherein the decontamination unit further comprising:
a controllable dehumidifier unit in fluid communication with a controllable air heater and humidity control unit to dehumidify the ambient air source to a selected humidity level;
an inlet filtration unit in fluid communication with the controllable dehumidifier unit to filter the ambient air; and
a controllable blower motor in fluid communication with the controllable dehumidifier unit to controllably draw the ambient air into the controllable dehumidifier unit and force the ambient air into the controllable air heater and humidity control unit.

16. The decontamination system of claim 14, wherein the decontamination unit further comprising:
a decontamination agent reservoir to hold the one or more decontamination agents; and
a controllable mixing valve for controlling a mixture of two or more decontamination agents.

17. The decontamination system of claim 14, wherein the decontamination unit further comprising:
user interface circuitry to enable a user to operate the system; and
power supply circuitry to supply power to at least the controllable air heater and humidity control unit, controllable vaporizer unit, and the controller circuitry.

18. The decontamination system of claim 14, wherein the decontamination unit further comprising:
a controllable pressure relief valve to maintain a selected pressure within the decontamination chamber; and
an exhaust port filtration unit to filer air exiting the decontamination chamber through the controllable pressure relief valve.

19. The decontamination system of claim 14, wherein the temperature and humidity of air within the decontamination chamber are controlled to be approximately 50 degrees Celsius and 50% humidity.

20. The decontamination system of claim 14, wherein the one or more decontamination agents are selected from an aqueous hydrogen peroxide solution and/or an aqueous ammonia solution.
